(19) United States
(12) Patent Application Publication
(10) Pub. No.: US 2002/0082592 A1
(43) Pub. Date: Jun. 27, 2002

(54) Coronary cutting, dilating, tamponading, and perfusing instrument

(76) Inventor: Banning Lary, Miami, FL (US)

Correspondence Address: Michael E. Klicpera, P.O. Box 573, La Jolla, CA, 92123, US

(21) Appl. No.: 10/057,541

(22) Filed: Jan. 24, 2002

Related U.S. Application Data
(60) Provisional application No. 60/124,768, filed on Mar. 17, 1999.

(51) Int. Cl. A61B 18/24; A61B 17/32

(52) U.S. Cl. 606/15; 606/194; 606/170

(57) ABSTRACT

The present invention is directed towards a method and device for cutting, dilating, tamponading a coronary vessel and with perfusion capabilities. The device consists of instrument general catheter having a distally position cylindrical member surrounded by a dilating balloon. The catheter is designed to be used with a guide wire for positioning the instrument and an advancement catheter permit the proper localization of the instrument inside a coronary artery. There are openings in a proximal cast member to permit the perfusion of blood which passes through the body of the instrument.

In the method of using the incising/dilating/tamponading/perfusing device, the first stage requires the employment of a guide wire that is passed through the area of stenosis in the coronary artery. The instrument is threaded over the guide wire to the stenosis and the distal protuberance performs initial dilation of the stenosis by apply proximal pressure to the advancement catheter. The cutting member is then positioned outside the distal end of the instrument. As the instrument is advanced, the cutting member progressively transects the stenosis and the artery while the rounded distal member progressively dilates the stenosis. With the artery completely incised longitudinally, the adventitia is transected, and the instrument is advanced over the length of the transection. The dilating balloon is distended to further dilate the now pliable artery and to tamponade the site of incision. It remains in this position while distal circulation is supplied by blood passing through the body of the instrument by entering and exiting the openings in the proximal and distal cast parts. Injections of contrast material and slow decompression of the dilating/tamponading balloon indicate when a firm clot has been established.

### PRIOR APPLICATIONS

This application claims the benefit of the Provisional Application Serial No. 06/124,768, filed on Mar. 7, 1999, the entire contents which are hereby incorporated by reference.

### FIELD OF THE INVENTION

The present invention relates to the field of angioplasty. In particular, the present invention relates to a coronary cutting, dilating, tamponading and perfusing catheter apparatus which provides dilation of the native lumen while simultaneously making a longitudinal transection of a coronary artery beneath the epicardium that results in a new conduit, all accomplished without blocking blood flow by use of a passive perfusion design apparatus.

### BACKGROUND OF THE INVENTION

It is well known that any significant reduction or restriction in the flow of blood through the arteries of the body can cause complications which may have serious ischemic consequences. Arterial blockages caused by plaque and fibrotic stenoses in coronary arteries are known to be a leading cause of heart attacks, subsequent strokes, and other debilitating maladies. Accordingly, it is extremely important for the health of a patient that any stenosis, or blockage, which is causing such a condition, be eliminated or reduced.

With the advent of bypass surgery techniques commonly known as CABG, the ischemic consequences of blockages in arterial segment can be alleviated by grafting around the lesion site a replacement means, typically with a saphenous vein graft. In this manner, blood is allowed to bypass the blockage in the affected artery and the blood supply to the body tissues downstream from the blockage is thereby restored. While bypass surgical procedures have become relatively safe, reliable, and effective, portions of the body must nevertheless be opened to accomplish the surgery. In other words, bypass surgery is invasive, and can consequently require significant post-operative recovery time. To avoid the drawbacks associated with invasive bypass surgery, less invasive surgical procedures have been developed wherein a device is inserted into the bloodstream of a patient and advanced into an artery to reduce or remove an arterial stenosis.

One well known and frequently used procedure to accomplish this task is popularly known as angioplasty. For a basic angioplasty procedure, a dilating balloon is positioned across the particular stenotic segment and the balloon is inflated to open the artery by breaking up and compressing the plaque which is creating the stenosis. The plaque, however, remains in the artery and is not removed. Unfortunately, in some cases, it appears that the plaque which remains in the artery may still present a stenosis. The removal of intra-arterial deposits are another common method for treating coronary atherosclerosis by mechanical means from a peripheral approach. However, with both of these interventional methods, the six month reoccurrence rate of restenosis can be 40% or more.

A further alternative treatment method involves percutaneous, intraluminal installation of one or more expandable, tubular stents or prostheses in sclerotic lesions. Stents or prostheses are known in the art as implants which function to maintain patency of a body lumen in humans and especially to such implants for use in blood vessels. They are typically formed from a cylindrical metal mesh which expand when internal pressure is applied. Alternatively, they can be formed of wire wrapped into a cylindrical shape.

Stents or prostheses can be used in a variety of tubular structures in the body including, but not limited to, arteries and veins, ureters, common bile ducts, and the like. Stents are used to expand a vascular lumen or to maintain its patency after angioplasty or atherectomy procedures, overlie an aortic dissecting aneurysm, tack dissections to the vessel wall, eliminate the risk of occlusion caused by flaps resulting from the intimal tears associated with primary interventional procedure, or prevent elastic recoil of the vessel.

These metallic stents are deployed inside an arterial segment and embedded in the vessel to maintain patency typically after angioplasty or atherectomy interventions. Once they are so positioned, they are extremely difficult to remove. Often the vessels in which they are placed become occluded or severely restenosed in a relative short period of time. These complications continue to occur the longer the stents remain in place, resulting in total or partial obstruction of blood flow through the artery. Usually, the distal portion of the artery will remain patent and is supplied by collateral circulation through branches of other major arteries. However, the decreased direct blood flow results in many cardiac problems. The use of stents after the interventional procedure or deploying a stent without any adjunctive procedure has decreased this rate to approximately 20% or less in the larger and more proximal arteries which are generally 3.0 mm or more in diameter. Yet, the use of stents have not completely solved the problem of restenosis, where hyperplasia growth sometimes occurs at the terminal ends of the stent. Furthermore, in smaller arteries less than 3.0 mm in diameter, the reoccurrence of a stenosis larger than 50% in relation to the segment diameter can again approximate 40% rate. Long term attrition with stents is not known and as yet there is no method to remove the stents. Brachytherapy and the injection or deposition of various genetic or bioactive materials, including radioactive sources, are currently being explored to decrease the reoccurrence rate. Furthermore, in approximately 30-60% of the vessels treated by angioplasty, there is a re-stenosis. This high recurrence rate is thought to be the result of fibrotic contraction in the lumen of the vessel.

It has been shown that when an angioplasty procedure is performed after the stenotic segment is longitudinally incised, the opening established through the segment is much larger as compared to standard angioplasty without the prior incisions. Still further, the increase in the opening in the stenotic segment is accomplished without tearing the vessel wall. Moreover, it has been found that incising the stenosis prior to dilation allows greater compression of the stenotic tissue with decreased likelihood of the stenosis rebuilding at a later date. As those skilled in the art will appreciate, the plaque creating a common arterial stenosis is somewhat fibrous and will tend to return to its original pre-dilation configuration. With this fibrous composition, the stenosis is therefore more likely to maintain a compressed configuration if the fibers are incised prior to balloon dilation. On the other hand, if the fibers in the stenosis is not incised first, the completeness of the compression of the stenosis is dependent on whether the inflated balloon is able to break apart fibers in the tissue as those skilled in the art will recognize, dilation of a segment is of course limited by the arteries able to withstand dilation. Over-dilation can have the catastrophic result of rupturing the vessel.

It is generally agreed that in consequent to the angioplasty procedure, it is the compression of the intra-arterial elements which results in restenosis by causing fibromyoendothelial hyperplasia. The compression which accompanies dilation is intensified by the encircling adventitia. This encasing structure can be stretched only minimally before it ruptures resulting in severe clinical outcomes.

One of the objects of the present invention and method is to provide a cutting device which, in cooperation with an angioplasty procedure, is able to produce an opening in a stenotic segment where the diameter of the opening is greater than the insertion diameter of the device.

Another object of the present invention and method is create a longitudinal transection of a coronary artery beneath the epicardium which will result in a new conduit.

It is also an object of the present invention to provide a device which allows improved control over the length of the incisions produced in the stenotic segment, and the depth of the incisions.

Yet another object of the present invention is to provide a device which is flexible enough to allow advancement of the device through narrow vessels and around sharp turns.

Still further, it is an object of the present invention to provide a device for longitudinally incising a stenotic segment of an artery which is relatively easy to manufacture and is comparatively economical.

Another object of the present invention is to tamponade a longitudinal cut in the arterial wall.

Still another object of the present invention is to continuously perfuse the distal artery and myocardium until firm clotting has been achieved.

### SUMMARY OF THE INVENTION

It has been demonstrated in several series of experiments that longitudinal transection of a coronary artery beneath the epicardium will result in a new conduit.

The treated vessel is then composed of the original arterial wall and another segment of vessel wall which originates on the blood clot and the maturing fibrosis. Such a procedure can be accomplished with minimal interruption of blood flow through the artery.

If the encircling constraint of the adventitia of the coronary artery is removed the artery can be dilated with minimal pressure to approach a normal diameter. Distal perfusion from the proximal to the distal artery can be supplied through the body of the instrument.

The present invention and associated method is directed to satisfy the prior defined needs. The device is used for cutting, dilating, tamponading a coronary vessel and has perfusion capabilities. The device consists of instrument having a cylindrical member or capsule surrounded by a dilating and tamponading balloon which in general, resembling a standard catheter assembly. The catheter is designed to be used with a guide wire for positioning the instrument and an advancement catheter permit the proper localization of the instrument inside a coronary artery. There are openings in a proximal cast member to permit the ingress of blood which passes through the body of the instrument. Engaged to the distal end of the tubular member is a distal cast part comprising; 1) an upper portion which contains the advancement catheter, the guide wire, and a transverse strut and 2) a lower partion which contains a chamber containing a deployment balloon, a cutting member.

In the method of using the incising/dilating/tamponading/perfusing device, the first stage requires the employment of a guide wire that is passed through the area of stenosis in the coronary artery. The instrument is threaded over the guide wire to the stenosis and the distal protuberance performs initial dilation of the stenosis by apply proximal pressure to the advancement catheter. The cutting member is then positioned outside the distal end of the instrument. As the instrument is advanced, the cutting member progressively transects the stenosis and the artery while the rounded distal member progressively dilates the stenosis. With the artery completely incised longitudinally, the adventitia is transected, and the instrument is advanced over the length of the transection. The dilating balloon is distended to further dilate the now pliable artery and to tamponade the site of incision. It remains in this position while distal circulation is supplied by blood passing through the body of the instrument by entering and exiting the openings in the proximal and distal cast parts. Injections of contrast material and slow decompression of the dilating/tamponading balloon indicate when a firm clot has been established.

These and other features, aspects, and advantages of the present invention will become better understood with regard to the following description, appended claims, and accompanying drawings.

### BRIEF DESCRIPTION OF THE FIGS.

FIG. 1. shows the incising/dilating/tamonading/perfusing device of the present invention in a coronary setting.

FIG. 2. shows a side view of the distal end of the incising/dilating/tamponading/perfusing invention in the disposed with an arterial segment, demonstrating the perfusion holes with both the cutting blade and balloon in a retracted position.

FIG. 3. shows a side cross-sectional view of the distal end of the incising/dilating/tamponading/perfusing invention, demonstrating the internal lumens with the cutting element in an extended position.

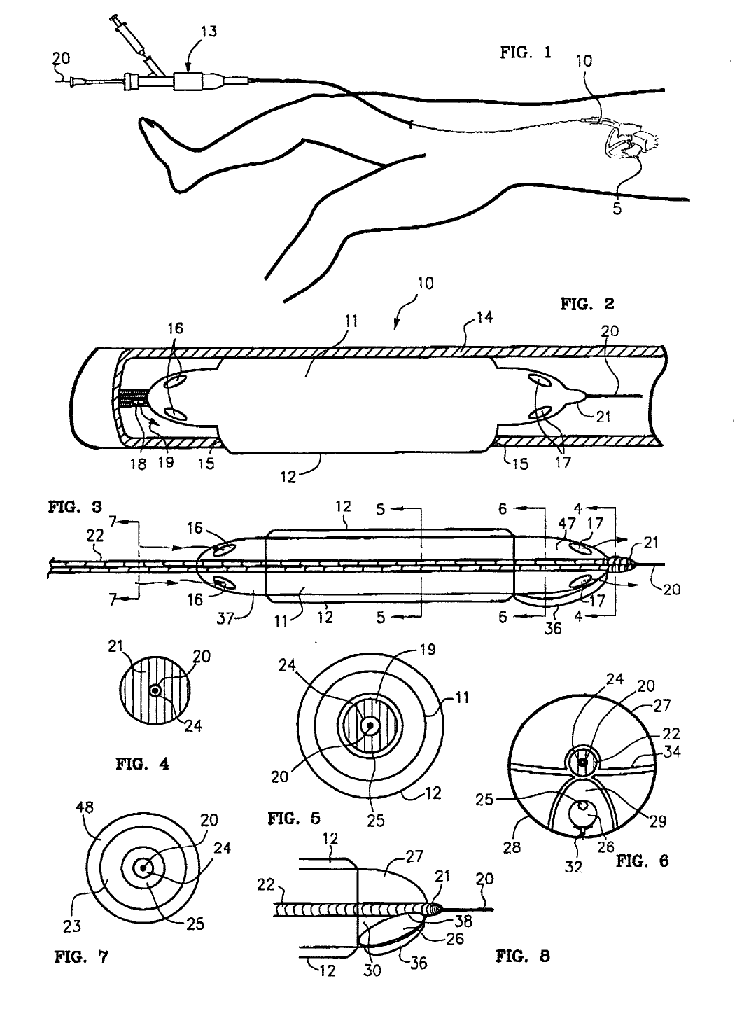

FIG. 4. shows a cross-sectional view of the distal protuberance of the invention, demonstrating the guide wire and guide wire lumen.

FIG. 5. shows a cross-sectional view taken from FIG. 2 demonstrating the tamponading balloon, distal member, and associated lumens.

FIG. 6. shows a cross-sectional view of the distal member demonstrating the upper cast part with advancement catheter, guide wire, and transverse struct and the lower cast part with a chamber embodying the deployment balloon and cutting member.

FIG. 7. shows a cross-sectional view of the catheter shaft, demonstrating the guide wire and guide wire lumen, balloon inflation/deflation lumen, and the cutting member deployment balloon inflation/deflation lumen.

FIG. 8. shows a saggital sectional view of the distal member detailing the distal protuberance, the distended deployment balloon, and externally positioned cutting member.

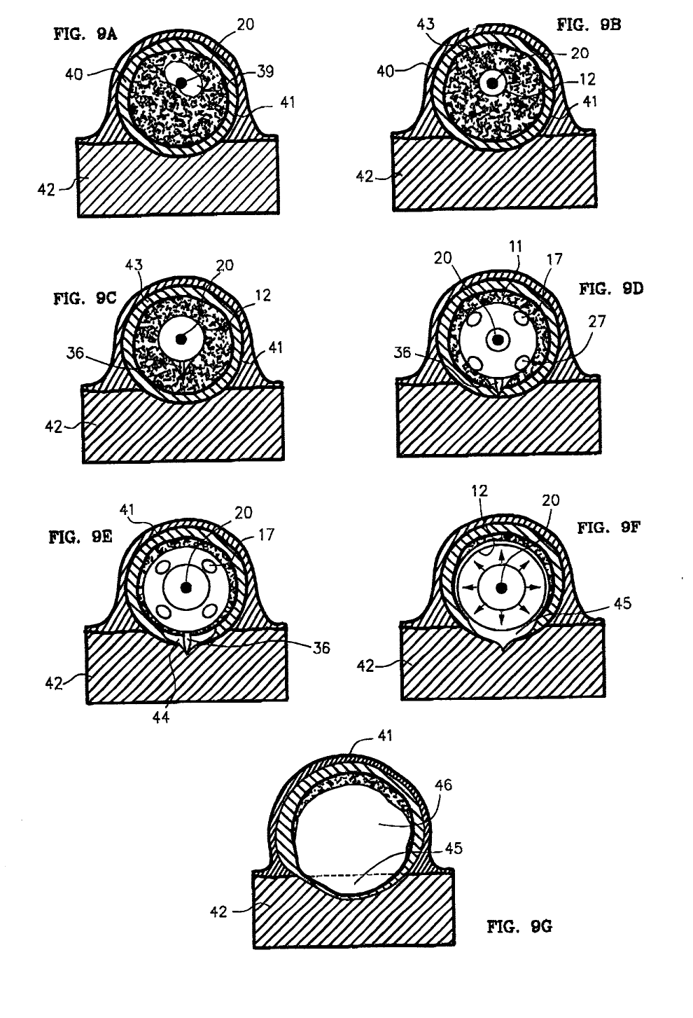

FIG. 9a. shows a cross-sectional view of the diseased artery with a guide wire placed in the narrowed lumen.

FIG. 9b. shows a cross-sectional view of the diseased artery with the incising/dilating/tamponading/perfusing device placed within the target segment demonstrating the initial dilating of the stenosis by the distal protuberance.

FIG. 9c. shows a cross-sectional view of the diseased artery with the incising/dilating/tamponading/perfusing device placed within the target segment demonstrating the cutting member in an extended position and initial stage of cutting the stenosis.

FIG. 9d. shows a cross-sectional view of the diseased artery with the incising/dilating/tamponading/perfusing device placed within the target segment demonstrating the perfusion capabilities of the device while further cutting of the stenosis is performed.

FIG. 9e. shows a cross-sectional view of the diseased artery with the incising/dilating/tamponading/perfusing device placed within the target segment demonstrating the cutting member transacting the adventitial constraint.

FIG. 9f. shows a cross-sectional view of the diseased artery with the incising/dilating/tamponading device demonstrating further dilation and tamponading of the incised arterial segment.

FIG. 9g. shows a cross-sectional view of the arterial segment treated with the incising/dilating/tamponading/perfusing device resulting with the artery dilated and the new conduit formed.

### DETAILED DESCRIPTION OF THE SPECIFIC EMBODIMENTS

FIG. 1. demonstrates the incising/dilating/tamonading/perfusing device of the present invention in a coronary setting using standard techniques for accessing and advancing the invention from a groin incision to the heart.

Now referring to FIG. 2, the present invention consists of instrument 10 having a cylindrical member or capsule 11 surrounded by a dilating and tamponading balloon 12, generally attached to the distal end of a typical advancement catheter 22. The catheter of the instrument is constructed of pliable smooth material such as various types of extruded polymeric materials. The cylindrical member, body or capsule 11 is engaged with a proximal 37 and distal 47 end sections, all of which could be constructed of smooth materials such as sintered steel or plastic. The catheter 22 is designed to be used with a guide wire 20 for positioning the instrument to permit the proper localization of the instrument inside a coronary artery 14. The guide wire is common to the industry, while the advancement member may have several configurations which will be flexible but capable of transmitting proximal pressures both continuous and/or intermittent. In addition, the advancement catheter may have a shorted guide wire lumen located at the distal end with a proximal opening to function as a rapid exchange design. Proximal openings 16 in the proximal cast member 37 permit the ingress of blood which passes through the body of the instrument to flow out of distal openings 17. The cylindrical body or capsule 11 has other openings or conduits which permit essential parts, such as the advancement catheter, contained guide wire and lumens for expanding the dilating balloon 12 and a deployment balloon 26. The advancement catheter 22 comprises a series of lumens running along the longitudinal length. One lumen in the advancement catheter connects a proximal port to distal catheter port 18 permitting the injection 19 of various substances including contrast media and medicaments. As demonstrated in this Figure and as used in clinical practice, the guide wire 20 is passed from a peripheral artery proximal through the area of stenosis in the coronary artery. The advancement catheter 22 and the distal instrument 10 follow the guide wire to the stenosis and the distal protuberance 21 performs initial dilation of the stenosis by apply proximal pressure or a hammering type action to the advancement catheter.

FIG. 3. shows a side or lateral cross-sectional view of the distal end of the incising/dilating/tamponading/perfusing invention, demonstrating the internal lumens and the cutting element 36 in an extended position. Also shown is the distal members construction, with proximal cast part 37 having openings 16 for the perfusion of blood, the central portion consisting of a cylindrical member or capsule 11, and the distal cast part 47 comprising an upper section 27 and a lower section 28, shown in more detail in FIG. 6. The central portion of the cylindrical body II is surrounded with a dilating and tamponading balloon 12 which is in fluid communication with a dilating/tamponading lumen 23 within the advancement catheter 22. As in the proximal cast part 37 of the cylindrical member or capsule 11, the distal cast parts have openings 17 for the expulsion (perfusion) of blood to the distal artery segment. As shown in the figure, the advancement catheter 22 transcends the cylindrical body 11 and terminates into a distal protuberance 21. Also extending though the advancement catheter 22 and cylindrical body 11 is a guide wire 20. As shown in this lateral view of the instrument, the cutting surface has assumed a position outside the distal end of instrument. As the instrument advances the cutting member progressively transects the stenosis and the artery while the rounded distal member progressively dilates the stenosis. With the artery completely incised longitudinally the adventitia has been transected, the instrument is advanced to position the length of the transection and the dilating balloon is distended to further dilate the now pliable artery and to tamponade the site of incision. It remains in this position while distal circulation is supplied by blood passing through the body of the instrument by entering and exiting the openings in the proximal and distal cast parts. Injections of contrast material and slow decompression of the dilating/tamponading balloon indicate when a firm clot has been established. At times a stent may be necessary either initially or later should the area of incision and dilation exceed expectations.

FIG. 4. shows a cross-sectional view taken from FIG. 2. of the distal protuberance of the invention, demonstrating the guide wire and guide wire lumen. The guide wire lumen 24 is positioned relatively in the center of the advancement catheter 22 for the guide wire 20 to pass. The position of the distal protuberance 21 functions to initiate the dilation. The advancement catheter 22 is firmly attached to the protuberance 21.

FIG. 5. shows a cross-sectional view taken from FIG. 2. demonstrating the dilating and tamponading balloon, cylindrical tubular member 11 and associated lumens.

FIG. 6 shows a cross-sectional view taken from FIG. 2. of the distal member demonstrating the upper cast part 27 with advancement catheter 22, guide wire 20, separated by a transverse strut 34 and the lower cast part 28 with a chamber 29 embodying the deployment balloon 26 and retracted cutting member 32. The distal upper cast part 27 is engaged to the advancement catheter 22 and the transverse strut 34. The distal lower cast part 28 contains a chamber 29 which embodies the deployment balloon 26, the retracted cutting member 32, and a conforming shelf 30. The upper 27 and lower distal cast parts can be joined by appropriate means, e.g. suitable adhesives, after the deployment balloon and cutting member are embodied or can be molded or extruded as a single structure. The transverse strut 34 seals the opening in the chamber containing those essential elements and when the two distal cast parts are joined, the transverse strut 34 strengthens the apparatus and closes the top of the open chamber which contains a previous place deployment balloon and its associated lumen or conduit 25. The top portion of the chamber 29 is initially open to permit placement of the deployment balloon 26 and the cutting member 32, but is closed by the approximation of the upper 27 and lower 28 cast parts. The lower cast part 28 has a cavity 29 for the positioning of the deployment balloon 26 and an port 38 engaged to its respective inflation/deflation lumen 25. The lower cast part 28 also has an opening 33 through which the cutting element 32 protrudes when the deployment balloon 26 is inflated. There is also a mechanism (not shown) which biases the cutting element 32 in the retracted or contracted position whenever the deployment balloon 26 in deflated. Blood exits openings 17 are in both the upper 27 and lower 28 distal cast parts.

FIG. 7. shows a cross-sectional view of the catheter shaft, demonstrating the positioning and relationship of the guide wire 20 and guide wire lumen 24, balloon inflation/deflation lumen 23, and the deployment balloon inflation/deflation lumen 25. The lumen 23 for the dilating/tamponading balloon 12, the lumen 25 for the deployment balloon 26, and the guide wire lumen 24 are created by the use of tubular materials common to the industry. The construction of the catheter can create the lumens by employing a coaxial design or alternately through the use of a multi-lumenal tubular structure. However, it is important that each of this lumens be independent of each other for each lumen functions to perform different and independent operations. Also, each lumen has its own proximal port located on the manifold adapter attached to the proximal end of the advancement catheter 22. Similarly, the dilating and tamponading balloon lumen 23 and deployment balloon lumen 25 is in fluid communication with its independent proximal port on the manifold and with the dilating and tamponading balloon 12 and deployment balloon 26, respectively.

FIG. 8. shows a saggital sectional view of the distal member detailing the distal protuberance 21, the distended deployment balloon 26, and externally positioned cutting member 36. The cutting member 32 (retracted) or 36 (extended) may have a number of configurations which would include a sharp edged blade, a serrated blade, a cautery, a harmonic scalpel, or a laser. The depth to which the cutting member is important and should be just enough to transect the entire arterial wall, after dilation, including the adventitia. Also shown is the advancement catheter 22 encircled with dilating/tamponating balloon 12 and engaged to the distal protuberance 21, both containing a guide wire 20. One or more ports 38 are in fluid communication with the deployment balloon lumen 25 in the advancement catheter 22 which functions to inflate and deflate the deployment balloon 26. FIG. 8. also shows the deployment balloon 26 in an inflated and distended configuration, with the externally positioned cutting member 36 and a conforming shelf 30 which seats the balloon.

FIG. 9a. shows a cross-sectional view of the diseased artery with a guide wire 20 placed in the narrowed lumen 39. The arterial segment depicted has a significant stenosis 40 surrounded by the adventitia 41. As depicted in FIG. 1, the general method of accessing the coronary vasculature is to create a puncture site in the patient's groin area and use one or more guide wires to access the particular coronary artery. Generally, a guiding catheter is employed to facilitate the advancement and placement of the present invention in the selected coronary artery.

FIG. 9b. shows a cross-sectional view of a diseased arterial segment with the incising/dilating/tamponading/perfusing device placed within the target segment. This figure also demonstrates that the distal protuberance 21 (not shown) is used to assist in the initial dilatation such that the present invention can be positioned in the stenosis 40.

FIG. 9c. shows a cross-sectional view of the diseased artery with the incising/dilating/tamponading/perfusing device placed within the target segment and demonstrating the cutting member 36 in an extended position and initial stage of cutting into the stenosis 40. In this stage of the clinical procedure, the cutting member 36 is extended by inflating the deployment balloon 26 with a fluid. Generally, the fluid used is a contrast medium or a mixed solution of contrast medium and physiologic saline. In this figure, the extended cutting member 36 has only penetrated a portion of the stenosis 40.

FIG. 9d. shows a cross-sectional view of the diseased artery with the incising/dilating/tamponading/perfusing device placed within the target segment demonstrating the perfusion capabilities of the device while further cutting of the stenosis is performed. The extended cutting member 36 has begun to transect the adventitia while maintaining perfusion capability.

FIG. 9e. shows a cross-sectional view of the diseased artery with the incising/dilating/tamponading/perfusion device placed within the target segment demonstrating the extended cutting member 36 fully transacting the adventitial constraint. Again, during this stage, perfusion capabilities are maintained.

FIG. 9f. shows a cross-sectional view of the diseased artery with the incising/dilating/tamponading/perfusion device demonstrating further dilation and tamponading of the incised arterial segment. However, at this stage, the deployment balloon 26 has been deflated by means of withdrawing fluid though the deployment balloon lumen 25 from a proximally positioned port. When the deployment balloon is deflated, the extended cutting member 36 is biased such that it becomes retracted into the distal lower cast part 28 thereby becoming a contracted cutting member 32.

FIG. 9g. shows a cross-sectional view of the arterial segment treated with the incising/dilating/tamponading/perfusing device resulting with the artery dilated and the new conduit formed. In the stage of the clinical procedure, the dilating and tamponating balloon has been deflated and the entire distal portion of the present invention is retracted from the treated arterial segment. Shown in this figure is the treated arterial segment 46 which has significantly reduced the stenotic area. The newly formed arterial conduit 45 is formed in the myocardium 42 as a result of the procedure. Bleeding form the longitudinally incised and dilated artery is contained by the epicardium and the periarterial tissues after the incising/dilating/tamponading balloon is slowly deflated whereby its collapses and can be removed from the treatment site.

I claim:

1. A device insertable into a vessel for dilating and tamponading a diseased segment comprising:
a catheter including a body portion, a proximal end, and a distal end, said catheter defining a longitudinal axis and an incising/dilating/tamponading member at said distal end, said catheter having one or more lumens;
said incising/dilating/tamponading member comprised of a tubular member surrounded by an expandable dilating balloon, a proximal member and a distal member, said proximal and distal members engaged to said tubular member; and
said distal member having an internal deployment balloon and an extendable cutting member.

2. The device as recited in claim 1 wherein said cutting member is designed to incise an arterial segment thereby creating a tamponade segment.

3. The device as recited in claim 1 wherein said cutting means is biased in a retracted position when said deployment balloon is deflated.

4. The device as recited in claim 1, wherein said incising/dilating/tamponading member has one or more proximal ports and one or more distal ports for the perfusion of blood.

5. The device as recited in claim 1, wherein said incising/dilating/tamponading member has perfusion capabilities.

6. The device as recited in claim 1, wherein said cutting member is comprised of a metallic material.

7. The device as recited in claim 1, wherein said cutting member is comprised of a rigid polymeric material.

8. The device as recited in claim 1, wherein said cutting member has a sharpened edge.

9. The device as recited in claim 1, wherein said cutting member has one or more teeth.

10. The device as recited in claim 1, wherein said cutting member has serrated blade.

11. The device as recited in claim 1, wherein said cutting member has a harmonic blade.

12. The device as recited in claim 1, wherein said cutting member consist of a laser.

13. The device as recited in claim 1, wherein said catheter includes a relatively short guide wire lumen located near the distal end of said catheter.

14. A method for treating a diseased arterial segment comprising:
advancing a catheter having an incising/dilating/tamponading member located at a distal end to said disease arterial segment;
extending a cutting element from said incising/dilating/tamponading member;
inflating a dilating/incising/tamponading balloon;
incising a portion of a stenosis and an adventitial barrier of said diseased segment with said cutting element;
creating a new conduit in myocardial tissues.

15. The method as recited in claim 14 wherein perfusion of blood is maintained.

16. The method as recited in claim 14 wherein said cutting element is retracted prior to forming the new conduit.

17. The method as recited in claim 16 wherein further dilation of said diseased segment is performed after said cutting element is retracted.

18. The method as recited in claim 14 wherein a distal protuberance performs an initial dilation of the diseased arterial segment as the catheter is advanced.

19. The method as recited in claim 14 wherein said cutting member incises an arterial segment thereby creating a tamponade segment.

20. A method for treating a diseased arterial segment comprising:
advancing a catheter having an incising/dilating/tamponading member located at a distal end to said disease arterial segment;
deploying a cutting element from said incising/dilating/tamponading member;
inflating a dilating/tamponading balloon thereby progressively incising a portion of a stenosis and an adventitial barrier of said diseased segment with said cutting element;
retracting said cutting element;
dilating said diseased arterial segment with said dilating/tamponading balloon;
forming a new conduit in myocardial tissues.

21. The method as recited in claim 20 wherein perfusion of blood is maintained.
